(19) United States
(12) Patent Application Publication
(10) Pub. No.: US 2005/0147203 A1
(43) Pub. Date: Jul. 7, 2005

(54) Reading imaging data

(76) Inventors: William Robert Ross, Scotia, NY (US); Ravinuthala Ramakrishna Rao, Bangalore (IN)

Correspondence Address: Berkeley Law and Technology Group, 680 NW Altishin Place, Beaverton, OR, 97006, US

(21) Appl. No.: 10/750,265

(22) Filed: Dec. 31, 2003

(51) Int. Cl. G21K 1/12; H05G 1/60; A61B 6/00; G01N 23/00

(52) U.S. Cl. 378/19

(57) ABSTRACT

Embodiments of methods, apparatus s, devices, and/or systems for reading imaging data are described.

### BACKGROUND

This disclosure is related to reading imaging data, such as, for example, for computed tomography (CT).

Fluoroscopic imaging, such as x-ray imaging, for example, may be used to create images of a human subject. One particular technique of fluoroscopic imaging is computed tomography (CT). A CT system typically operates by emitting radiation from a source, such as an x-ray source, towards a subject, such as a human subject. Typically, the x-rays are emitted from multiple angles with reference to the subject, resulting in several projections from differing angles of incidence. A portion of the radiation may be absorbed or deflected by the subject, and a portion may pass through the subject, and be ultimately detected either directly or indirectly, by a set of detector elements. The detector elements may, for example, produce a digital signal, and may provide the signal to a computing device. The computing device may be configured to construct the one or more signals into a virtual three-dimensional image of the subject, depending, for example, on the number of projections produced. However, obtaining multiple projections may be computationally complex and time consuming. A need exists, therefore, for improved methods and apparatuses for imaging, such as for CT, for example.

### BRIEF DESCRIPTION OF THE DRAWINGS

Subject matter is particularly pointed out and distinctly claimed in the concluding portion of the specification. The claimed subject matter, however, both as to organization and method of operation, together with objects, features, and advantages thereof, may best be understood by reference of the following detailed description when read with the accompanying drawings in which:

FIG. 1 is a schematic diagram of an embodiment of an imaging system;

FIG. 2 is a schematic diagram of an embodiment of an imaging scanner;

FIG. 3 is a schematic diagram illustrating several potential embodiments or techniques to read imaging data from an imaging detector panel;

FIG. 4 is a schematic diagram illustrating another potential embodiment or technique to read imaging data from an imaging detector panel; and

FIG. 5 is a flowchart illustrating one embodiment of a method of reading imaging data.

### DETAILED DESCRIPTION

In the following detailed description, numerous specific details are set forth to provide a thorough understanding of the claimed subject matter. However, it will be understood by those skilled in the art that the claimed subject matter may be practiced without these specific details. In other instances, well-known methods, procedures, components and/or circuits have not been described in detail so as not to obscure the claimed subject matter.

Fluoroscopic imaging, such as x-ray imaging, for example, has become a usyeful tool for numerous applications. Although particular embodiments of the claimed subject matter will be described with reference to medical applications, it is worthwhile to note that numerous non-medical applications of imaging exist, and the claimed subject matter is not limited in this respect One particular imaging technique that may be used in medical imaging is computed tomography (CT). One particular type of CT comprises volumetric computed tomography (VCT). VCT is a technologically advanced variation of two-dimensional imaging, where a representative three-dimensional volumetric image may be constructed based at least in part on a plurality of two-dimensional images. Such two-dimensional images may comprise digital images based at least in part on one or more sets of data acquired through radiological imaging using relatively low doses of radiation, in at least one embodiment. In one embodiment, a computed tomography system may comprise a CT scanner A CT scanner, in this embodiment, operates by projecting fan shaped or cone shaped beams, such as x-ray beams, from a source, such as an x-ray source. These x-ray beams are projected towards a subject, and a portion of the x-ray beams may be attenuated, such as by absorption or scattering, for example. A portion of the x-ray beams that are not attenuated may strike a scintillator that produces photons detected by a detector, which may comprise multiple flat panel detectors, for example. If the CT scanner is configured to obtain multiple projections, the source and/or the detector may take various positions or locations with reference to the subject in order to obtain a variety of projections. A series of projections, which may also be referred to as images or views, may be obtained, such as a series of projections of the subject obtained from differing angles of incidence. In one embodiment, a single constructed projection may be referred to as a slice, and multiple slices may be employed to construct a three dimensional image of the subject, for example.

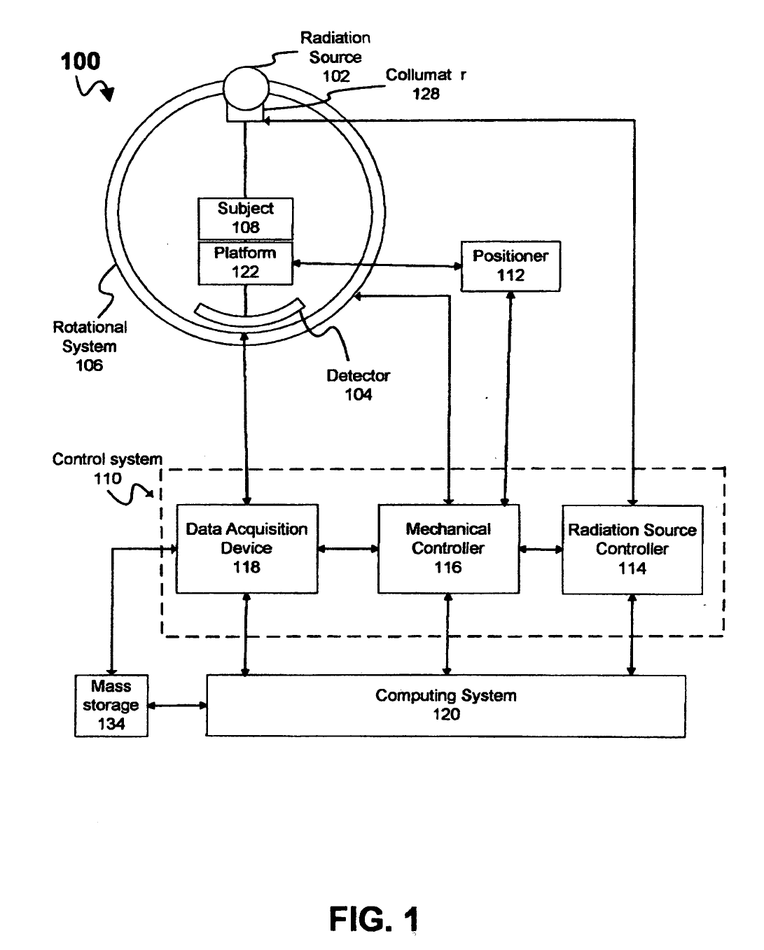

Referring now to FIG. 1, there is illustrated a schematic representation of an embodiment of a CT system, although, of course, the claimed subject matter is not limited in scope to this particular system, to a particular system, or necessarily even to CT. However, in FIG. 1, system embodiment 100 comprises at least one radiation source 102, which may be an x-ray source, although it is worthwhile to note that other imaging sources may be employed in addition to radiation sources. However, for this particular embodiment, a source capable of producing penetrating photons, for example, may be used. In one particular embodiment, radiation source 102 may be a source capable of producing x-ray spectra, such as a 26 kV 100 mAs Rhodium filtered beam from a Rhodium anode. However, the claimed subject matter is not limited in this respect. Filter materials suitable for use in a radiation imaging system such as described may include Molybdenum, Rhodium, Copper or Aluminum, as just a few examples. Additionally, anode materials may include Molybdenum, Rhodium or Tungsten, as just a few examples. However, as previously indicated, non-radiation based imaging systems are also included within the scope of the claimed subject matter.

Here, system 100 further comprises a collimator 128, which may be configured to control the range of a beam of radiation, and detector 104, which may comprise a detector array, including a series of panels, for example. In this embodiment, radiation source 102 and detector 104 may be coupled to a rotational system 106, which may comprise a gantry, and one or more mechanical components (not shown). The rotational system 106 may be configured to rotate radiation source 102 and detector 104 in a particular or selected manner with respect to platform 122 and subject 108, for example. Platform 122, which may comprise a patient platform, for example, may be configured to adjust the position of subject 108 relative to the source and detector, for example, and may be controlled by positioner 112.

CT system 100 may further comprise a control system 110. Control system 110, in this particular embodiment, may comprise a radiation source controller 114, a mechanical controller 116 and a data acquisition device 118. Additionally, a computing system 120 and mass storage 134 may be configured to exchange electronic data with one or more components of control system 110, for example. It is noted, however, that this is merely one particular embodiment of an imaging system, such as a tomography system, and the claimed subject matter is not limited to this particular embodiment.

In operation, CT system 100 may obtain one or more projections of a subject 108 in the following manner, although, it is worthwhile to note that this is just one embodiment, and the claimed subject matter is not so limited. Radiation source controller 114 may provide signals, such as timing and/or control signals, to radiation source 102, driving it to emit radiation for a particular period of time. Radiation source 102 may, depending at least in part on the type of radiation source, produce a stream of photons, which may comprise a conical or fan shaped stream of photons, for example, and may be directed by collimator 104, for example. The stream of photons may be substantially directed towards subject 108, for example. Depending on factors such as the matter density of subject 108 and the angle of incidence of the emitted radiation with respect to subject 108, photons may be scattered, attenuated, unattenuated, or absorbed, for example. In this particular embodiment, the photons may strike a scintillator which may, as a result, produce light at least a portion of which may be capable of being measured by pixels of detector 104. One or more photons may therefore strike one or more detector elements, such as pixels (not shown) of detector 104, and one or more of pixels of detector 104 may have incident upon it a light signal representing a particular photon count or pixel intensity. The pixels of detector 104 may produce electrical signals based at least in part on the incident signal detected, for example, and at least a portion of the electrical signals may be provided to a device capable of processing the electrical signals, such as computing system 120, for example. The electrical signals may be subsequently processed by computing device 120, and an image of the scanned area of subject 108 may be produced based at least in part on the electrical signals, for example.

In this embodiment, if differing projections are to be obtained, mechanical controller may instruct rotational system 106 to rotate radiation source 102 and detector 104 in a particular or selected manner with respect to platform 122 and subject 108, for example. Additionally, positioner 112 may adjust patient platform 122, for example, to adjust the position of subject 108 relative to the source and detector, for example. Thus, one or more additional projections may be obtained by performing one or more of the aforementioned operations. The desired number of additional projections may depend on a variety of factors, such as the complexity of the subject to be imaged, type of imaging source, and/or the complexity of the imaging system, for example. Of course, the claimed subject matter is not limited to this particular technique for obtaining additionally projections. Depending on the number of projections obtained, computing system 120 may at least partially construct an image, such as a three-dimensional image, from a plurality of projections. Technology to construct a three-dimensional image from one or more two-dimensional images is well-known, and will not be discussed in detail, in order to conserve space. The reconstructed image may be stored in a mass storage device 134, for example. Additionally, although not illustrated in detail, computing system 120 may comprise one or more microprocessors, one or more memory devices, one or more input/output ports, and/or a user interface, such as a graphical user interface (GUI), for example. The user interface may be utilized to provide one or more instructions to portions of CT system 100, for example, via computing system 120. For example, a user may provide scanning parameters and instructions to the CT system 100 by use of a user interface, for example. Additionally, an output device, such as an LCD screen, as one example, may be used to at least partially display the image. Again, this is one particular embodiment, and the claimed subject matter is not limited to this particular example. A variety of imaging systems are possible and included within the scope of the claimed subject matter.

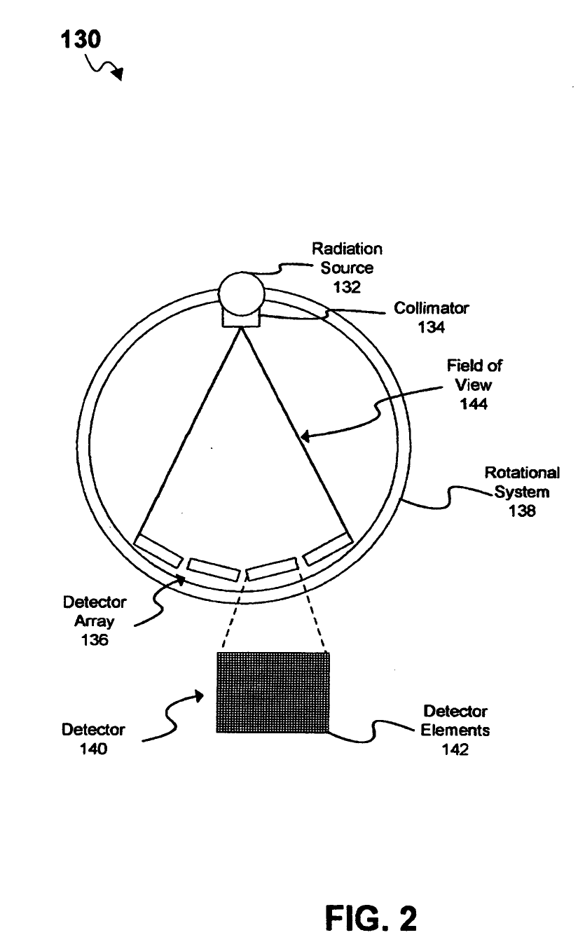

Referring now to FIG. 2, there is illustrated an embodiment of a CT scanner, here 130, which may comprise a portion of a CT system, such as CT system embodiment 100 of FIG. 1, for example. As previously explained, this is simply one particular embodiment of an imaging system. However, CT scanner embodiment 130 is illustrated as comprising a radiation source 132, a collimator 134, a detector array 136, and a rotational system 138. As stated previously, a system such as illustrated may be configured to provide one or more fluoroscopic projections of a subject. In this embodiment, radiation source 132, which may comprise an x-ray source, and detector array 136 may be fixed to rotational system 138, which may be comprised of a gantry and one or more mechanical components (not shown). In this particular embodiment, detector array 136 comprises an array of flat panel detectors, which may comprise amorphous silicon panels, comprising multiple arrays of photodiodes formed from photolithography, although, of course, the claimed subject matter is not necessarily limited in scope in this respect. For this particular embodiment, detectors 140 of detector array 136 may be arranged to be immediately adjacent to one another, and subtending a portion of an arc formed at least in part by rotational system 138, for example, although, in alternative embodiments, one or more detectors may not necessarily be adjacent, and it is worthwhile to note that the claimed subject matter is not limited to any particular detector configuration.

In one embodiment, detectors 140 comprise a plurality of detector elements 142, which may comprise pixels, arranged in multiple arrays, such as a rectilinear array comprising 1024×1024 pixels, for example, although this is just one example embodiment. The detector elements, such as detector element 142, for example, may comprise a pixel having a pixel pitch of approximately 1 millimeter, and may further comprise a photodiode, configured to detect incident photons, and provide electrical signals based on the charge produced across the diode, for example. Additionally, a scintillator (hot shown) may be coupled to one or more detectors 140, and may be configured to generate light when struck by x-rays, which may then be detectable by the photodiodes, for example. In one example embodiment, CT scanner 130 comprises an array of flat panel detectors, where the detectors comprise 20 centimeter by 20 centimeter flat panel x-ray detectors comprising a 1024 by 1024 rectilinear grid of photodiodes, which may provide an isotropic resolution of about 200 microns, for example, although, again, the claimed subject matter is not limited in scope in this respect.

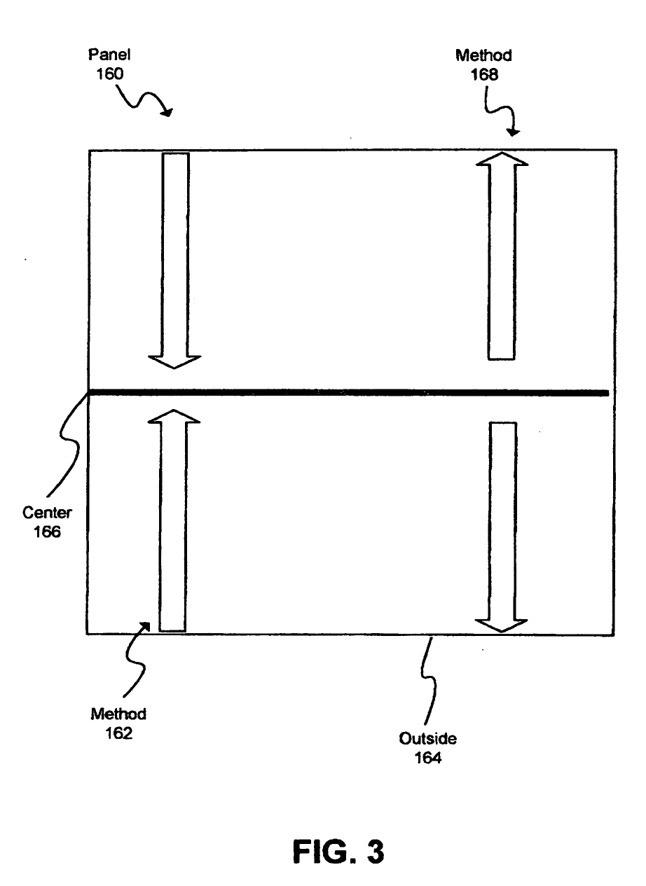

In operation, CT scanner 130 may operate to obtain one or more projections in the following manner: Radiation source 132 may emit x-rays, and collimator 134 may cause the x-rays to be projected in a particular field of view 144. At least a portion of the x-rays may result in light detected by one or more pixeIs 142 of one or more detectors 140, such as after striking a scintillator, for example. The pixels of one or more detectors 140 may be read out, which may comprise digitization and providing the digitized signal to a computing system, for example. In one particular embodiment, rows of pixels may be read out serially, for example. In another embodiment, pixel rows at opposing ends of a panel may be read out serially. These embodiments may be better understood with reference to FIGS. 3 and/or 4, explained in more detail later.

In this embodiment, the elapsed time for emission of x-rays, and the time elapsed for a full read out of the detectors 140 may be referred to as the frame rate, in at least one embodiment. As stated previously, the number of projections may vary, but, in one embodiment, a volumetric computed tomography system may acquire on the order of approximately 1000 projections, for example, in order to construct a multidimensional image. This acquisition of 1000 projections or thereabouts, using a full read out of the associated detectors, may take approximately 30 seconds. However, for a variety of reasons, 30 seconds may be longer than desired. For example, it may be undesirable to expose a subject to radiation for that amount of time in an imaging system where radiation is employed. Likewise, such an amount of time may be undesirably inconvenient for a subject to attempt to remain still in order to obtain a high quality image.

Therefore, as suggested previously, it may be desirable to reduce the time involved, such as by reducing the frame rate, for example. In one embodiment, the frame rate may be reduced by selectively limiting the read out of detector elements, rows of dectector elements, and/or of grouped rows of elements. This aspect of the frame rate of a panel, such as panel 140, may be better understood with reference to FIG. 3.

Illustrated in FIG. 3 are two differing techniques or embodiments for reading data from a detector panel, such as panel 160. Of course, the claimed subject matter is not restricted to either technique. Method embodiment 162 illustrates the read out of panel 160 by dividing the panel into two half sections, and reading out directly opposing rows of pixels beginning from the most remote rows of the panel, e.g., 164, to the center, e.g., 166, of the panel. Method embodiment 168 illustrates the read out of panel 160 by also dividing the panel into two half sections; however, instead, rows of pixels are read from the center of the panel to the most remote rows. As will be explained in more detail hereinafter, method embodiment 168 may have an advantage by providing improved frame times, such as when used by a CT system, for example, CT system 100 of FIG. 1.

As stated previously, detector panels may be capable of being read out serially, such as by substantially simultaneously reading directly opposing rows of detector elements. Thus, the detector elements of the opposing rows are read in parallel, but different sets of opposing rows are read out serially. Detector panels may be equipped with circuitry that provides for reading out data in this manner, such as duplicate circuitry, for example. Read out of detector panels may be controlled by use of firmware coupled to the detector panel, such as one or more field programmable gate arrays (FPGA) which control one or more read out operations, for example, although, of course, the claimed subject matter is not limited in this respect.

However, referring again to FIG. 2, depending on the field of view of an x-ray source, and/or the dimensions of a subject, one or more pixels or even rows of pixels of a detector panel may not contain relevant data. In such circumstances, reading out every pixel of a detector, therefore, may not provide any additional data usable for imaging. In other words, the projected photons may not project on the entire panel of a detector. If this is the case, then reducing the number of pixels read may reduce the frame rate without a corresponding degradation in the resulting image. Likewise, the time to construct an image may additionally be reduced. Alternatively, even where pixels contain data, depending upon pixel location, the value of the data may be relatively small so that, again, read out may be omitted to reduce frame time.

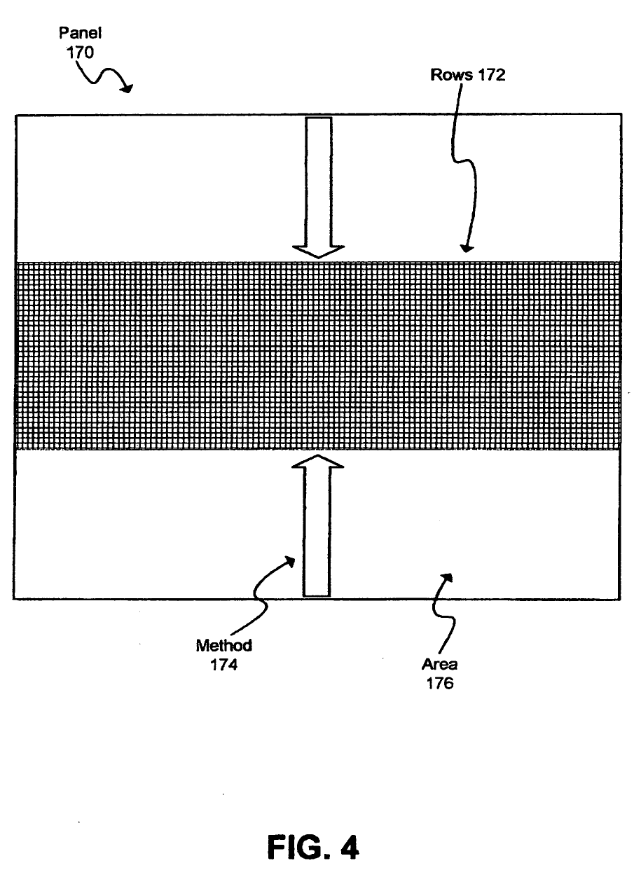

In this embodiment, and referring to FIG. 4, there is illustrated a flat panel detector 170. In this embodiment, multiple rows 172 may contain projection data, and other rows of panel 170 may not contain relevant projection data. Thus, a read out of the panel, in this embodiment, may comprise a selective read out of only the pixels contained in rows 172, for example, and, as a result, the read out process will take less time, if there are rows of the panel that are not read out, for example. In this embodiment, rows 172 may be referred to as an area of interest or a determined area of interest. The area of interest, in one embodiment, may be specified by a user, for example. Alternatively, the area of interest may be determined by a computing system, such as by determining what portion of a panel contains an area of interest based on subject size, for example, or the area of interest could be determined by a resident device of a panel, such as the aforementioned FGPA, for example. These, of course, are merely examples and are in no way intended to limit the scope of the claimed subject matter. There may other ways of determining the area of interest not specified here that are nonetheless included within the scope of the claimed subject matter. In this embodiment, the determined area of interest may also be read by applying the technique of method embodiment 168 of FIG. 3, for example. Thus, such rows may be read from the center of the panel to the more remote rows for such an embodiment. Where this occurs, again, the more relevant data is read out without waiting to read less relevant or irrelevant data, again, potential reducing frame time.

In another embodiment, one or more pixels of rows, such as two or more rows of pixels, for example, may be grouped, or ganged, and may be read out as if the multiple rows comprise a single row. For example, two or more rows of panel 170, in this context, may be grouped or ganged. As a result of such a process, effectively a mathematical combining of pixel signal values, such as by averaging adjacent pixels of the multiple rows, to provide a single row may result.

It is noted that embodiments may also be combined to permit a variety of tradeoffs to be made, depending upon the particular context. For example, frame time may be reduced by ganging or grouping rows as described. Alternatively, for a relatively fixed frame time, ganging or grouping rows may permit a larger area of interest to be imaged. Of course, ganging or grouping of rows as described will typically reduce resolution, however. It is also noted that data may be read from separate panels substantially concurrently, thereby reducing frame time.

It is noted that in circumstances where the entire panel of pixels may not be read out, one or more rows of pixels of a panel not residing in the area of interest may have detected one or more photons, and, as a result, may have an electrical charge. By not reading out these particular pixels, a charge may accumulate after multiple projections are obtained, and may potentially result in damage to the panel. Thus, it may be desirable, at times, to read out the pixels of rows not in the area of interest. In such a manner, the pixels of such rows may be ‘scrubbed.’ In this context, scrubbed refers to reading out a portion of the pixels of a panel, and thereby substantially discharging the pixels, regardless of whether these pixels are within an area of interest, for example. It is noted that scrubbing may take place in groups or gangs of rows to reduce the amount of time it may take to accomplish.

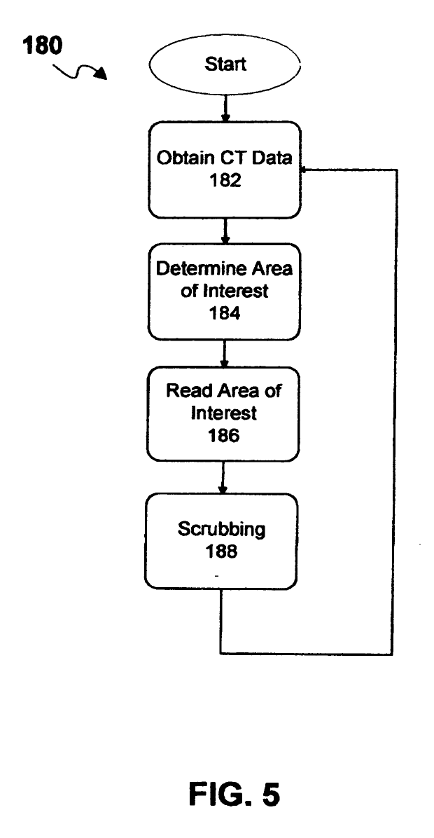

Referring now to FIG. 5, a flowchart depicts another potential embodiment, which may be used to at least partially operate a CT system, in this particular example. The operations of flowchart 180 illustrated in FIG. 5 may be perform on a CT system, such as system 100 of FIG. 1, for example, although the claimed subject matter is not limited in this respect, and the order in which the particular operations are presented does not necessarily limit the claimed subject matter to any particular order. Likewise, intervening operations not illustrated in FIG. 5 may also be employed in an embodiment.

Embodiments of block diagram 180 depicted in FIG. 5 may be implemented in software, hardware and/or firmware, and may comprise discrete operations, which may be well known to those of skill in the art. In this embodiment, radiological data for a subject may be obtained by one or more rows of pixels of a CT system, as illustrated at block 182. At block 184, an area of interest may be determined. The area of interest may be substantially read outs at block 186. At block 188, one or more pixels of one or more rows, such as one or more rows not within the area of interest or not read out at block 186, for example, may be scrubbed. As described previously, this may comprise the reading out of a portion of the pixels of a panel, for example, and thereby result in substantially discharging the pixels. The one or more pixels of rows scrubbed may be grouped or ganged, as previously described. Where multiple projections are desired, many iterations of flowchart 180 may take place.

In this embodiment, at block 184, an area of interest may comprise an area less than the entire projected image of a subject, for example, or may comprise the entire area wherein data is obtained relating to a subject, but still less than an entire panel, for example. However, in another embodiment, less than all the data obtained relative to a subject may comprise the area of interest, depending on the particular context. In one embodiment, the determination of an area of interest may be determined by a user, such as a radiologist, and may be based at least in part on the particular area of a subject for which the user would like to construct an image, for example. Alternatively, the area of interest may be determined by a computing device or a device resident on a detector, as stated previously. This determination may be additionally based on the size of a subject, for example, or may be based on the desired resolution of a subject. Thus, in this context, any one of a virtually infinite variety of factors and techniques may be employed to determine or at least partially determine the area of interest.

In this embodiment, at block 186, read out of the area of interest may comprise providing electrical signals, such as digitized data, to a data acquisition device. The read out, in one embodiment, may comprise a parallel reading of electrical data for two opposing rows of pixels of the area of interest, for example. Thus, different sets of rows may be read serially, as previously described. In one embodiment, the read out may begin at or towards the center of the area of interest and may proceed in an outward fashion, as previously described. In this particular embodiment, at block 188, scrubbing may comprise grouping or ganging of one or more pixels, such as rows of pixels outside the area of interest, for example, or grouping on or mor pixels not read at functional block 186, for example, and reading out the grouped rows of pixels. In such an embodiment, electrical signals obtained by reading out the grouped rows of pixels may not be stored, but may be read out and discarded, for example. As stated previously, this may result in the scrubbing of a substantial portion of a detector panel, for example. In an alternative embodiment, of course, the rows outside the area of interest may be scrubbed a row at a time, but that has a disadvantage of taking a greater amount of time to complete.

It is, of course, now appreciated, based at least in part on the foregoing disclosure, that software may be produced capable of performing one or more of the above-described functions. It will, of course, also be understood that, although particular embodiments have just been described, the claimed subject matter is not limited in scope to a particular embodiment or implementation. For example, one embodiment may be in hardware, such as implemented to operate on a device or combination of devices, as previously described, for example, whereas another embodiment may be in software. Likewise, an embodiment may be implemented in firmware, or as any combination of hardware, software, and/or firmware, for example. Likewise, although the claimed subject matter is not limited in scope in this respect, one embodiment may comprise one or more articles, such as a storage medium or storage media. This storage media, such as, one or more CD-ROMs and/or disks, for example, may have stored thereon instructions, that when executed by a system, such as a computer system, computing platform, or other system, for example, may result in an embodiment of a method in accordance with the claimed subject matter being executed, such as one of the embodiments previously described, for example. As one potential example, a computing platform may include one or more processing units or processors, one or more input/output devices, such as a display, a keyboard and/or a mouse, and/or one or more memories, such as static random access memory, dynamic random access memory, flash memory, and/or a hard drive, although, again, the claimed subject matter is not limited in scope to this example.

In the preceding description, various aspects of the claimed subject matter have been described. For purposes of explanation, specific numbers, systems and/or configurations were set forth to provide a thorough understanding of the claimed subject matter. However, it should be apparent to one skilled in the art having the benefit of this disclosure that the claimed subject matter may be practiced without the specific details. In other instances, well-known features were omitted and/or simplified so as not to obscure the claimed subject matter. While certain features have been illustrated and/or described herein, many modifications, substitutions, changes and/or equivalents will now occur to those skilled in the art. It is, therefore, to be understood that the appended claims are intended to cover all such modifications and/or changes as fall within the true spirit of the claimed subject matter.

1. A method comprising:
obtaining imaging data in a detector panel;
determining an area of interest for said panel, wherein said area of interest includes a portion of the obtained imaging data, wherein said panel comprises multiple pixel rows, said area of interest comprising more than two and less than all of said multiple pixel rows of said panel; and
reading at least a portion of said imaging data, wherein said portion comprises less than all the obtained imaging data and at least a portion of said data obtained in said determined area of interest.

2. The method of claim 1, wherein said determined area of interest comprises less than half of all of said multiple pixel rows of said panel.

3. The method of claim 1, wherein said panel is divided with one half of said multiple pixel rows on one half of said panel and another half of said multiple pixel rows on a remaining half of said panel.

4. The method of claim 3, wherein said determined area of interest is also divided with one half on half of said panel and another half on a remaining half of said panel.

5. The method of claim 4, wherein data in said determined area of interest is read in parallel in multiple immediately adjacent rows.

6. The method of claim 4, wherein data in said determined area of interest is read by row, starting from the center of said panel and reading towards rows remote from said center.

7. The method of claim 4, wherein said data is read from directly opposing rows in parallel.

8. The method of claim 1, and further comprising:
determining said area of interest based at least in part on the dimensions of a subject to be imaged.

9. The method of claim 1, wherein data from separate panels are read substantially concurrently.

10. The method of claim 1, and further comprising scrubbing said panel by reading out data from rows outside said determined area of interest.

11. The method of claim 10, wherein said scrubbing takes place a group of adjacent rows at a time.

12. A method of tomographic imaging, comprising:
emitting radiation from one or more radiation sources towards a subject situated in front of a set of panel detectors;
as a result of said emitted radiation, at least a portion of at least some of said set of panel detectors having stored data related to said subject;
reading less than all of said stored data related to said subject from said set of panels wherein reading less than all of said stored data comprises:
determining an area of interest, wherein said area of interest comprises an area of said set of panel detectors less than the entire area of said set of panel detectors: and
reading at least a portion of the data contained in said determined area of interest.

13. The method of claim 12, wherein said one or more radiation sources comprises one or more x-ray sources.

14. The method of claim 12, wherein said panel detectors comprises flat panel amorphous silicon panels.

15. (canceled)

16. A method of tomographic imaging, comprising:
emitting radiation from one or more radiation sources towards a subject situated in front of a set of panel detectors so that only a portion of at least some of said set of panel detectors are able to store signals related to said subject;
reading said stored signals related to said subject from said portion of at least some of said set of panel detectors, wherein reading said stored signals comprises:
determining an area of interest, wherein said area of interest comprises an area of said set of panel detectors less than the entire area of said set of panel detectors; and
reading said stored signals contained in said determined area of interest.

17. The method of claim 16, wherein said one or more radiation sources comprises one or more x-ray sources.

18. The method of claim 16, wherein said panel detectors comprises flat panel amorphous silicon panels.

19. (canceled)

20. A tomography system, comprising:
a least one radiation source;
a detector array;
a computer coupled to said detector array, said computer configured to, in operation:
obtain imaging data in said detector array:
determine an area of interest for said detector array, said determined area of interest being less than said entire detector array; and
read data from said determined area of interest.

21. The tomography system of claim 20, wherein said radiation source comprises an x-ray source.

22. The tomography system of claim 20, wherein said detector array comprises an array of flat panel amorphous silicon detectors.

23. The tomography system of claim 20, wherein said system is capable of producing radiological images of a human subjects.

24. The tomography system of claim 20, said computer is further configured to, in operation:
determine said area of interest based at least in part on the dimensions of said subject.

25. The tomography system of claim 20, wherein said computer is further configured to, in operation:
scrub at least the portion of said detector array outside said determined area of interest.

26. An article comprising: a storage medium having stored thereon instructions that, when executed, results in a method of reading imaging data stored in a detector panel:
determining an area of interest for said panel, wherein said area of interest includes a portion of the stored imaging data, wherein said panel comprises multiple pixel rows, said area of interest comprising more than two and less than all of said multiple pixel rows of said panel; and
reading at least a portion of said imaging data, wherein said portion comprises less than all the stored imaging data and at least a portion of said data stored in said determined area of interest.

27. The article of claim 26, wherein said panel is divided with one half of said multiple pixel rows on one half of said panel and another half of said multiple pixel rows on a remaining half of said panel.

28. The article of claim 27, wherein said determined area of interested is also divided with one half on half of said panel and another half on a remaining half of said panel.

29. The article of claim 28, wherein said instructions, when executed, further result in said data in said determined area of interest being read in parallel in multiple immediately adjacent rows.

30. The article of claim 28, wherein said instructions, when executed, further result in said data in said determined area of interest being read by row, starting from the center of said panel and reading towards rows remote from said center.
